(19) United States
(12) Patent Application Publication
(10) Pub. No.: US 2004/0229840 A1
(43) Pub. Date: Nov. 18, 2004

(54) Nucleoside derivatives as inhibitors of RNA-dependent RNA viral polymerase

(76) Inventors: Balkrishen Bhat, Carlsbad, CA (US); Anne B. Eldrup, Encinitas, CA (US); Steven S. Carroll, Yardley, PA (US); David B. Olsen, Lansdale, PA (US); Daniel R. McMasters, New York, NY (US); Malcolm MacCoss, Freehold, NJ (US); Jie Xia, Carlsbad, CA (US)

Correspondence Address: MERCK AND CO INC, P O BOX 2000, RAHWAY, NJ, 070650907

(21) Appl. No.: 10/694,677

(22) Filed: Oct. 28, 2003

Related U.S. Application Data
(60) Provisional application No. 60/422,045, filed on Oct. 29, 2002.

(51) Int. Cl. A61K 31/7072; A61K 31/513; C 07D 4 9/04

(52) U.S. Cl. 514/50; 514/269; 536/28.1; 544/317

(57) ABSTRACT

The present invention provides nucleoside compounds and certain derivatives thereof which are inhibitors of RNA-dependent RNA viral polymerase. These compounds are inhibitors of RNA-dependent RNA viral replication and are useful for the treatment of RNA-dependent RNA viral infection. They are particularly useful as inhibitors of hepatitis C virus (HCV) NS5B polymerase, as inhibitors of HCV replication, and/or for the treatment of hepatitis C infection. The invention also describes pharmaceutical compositions containing such nucleoside compounds alone or in combination with other agents active against RNA-dependent RNA viral infection, in particular HCV infection. Also disclosed are methods of inhibiting RNA-dependent RNA polymerase, inhibiting RNA-dependent RNA viral replication, and/or treating RNA-dependent RNA viral infection with the nucleoside compounds of the present invention.

### CROSS-REFERENCE TO RELATED APPLICATIONS

The present invention is related to U.S. provisional application Ser. No. 60/422,045, filed Oct. 29, 2002, the contents of which are hereby incorporated by reference.

### FIELD OF THE INVENTION

The present invention is concerned with nucleoside compounds and certain derivatives thereof, their synthesis, and their use as inhibitors of RNA-dependent RNA viral polymerase. The compounds of the present invention are inhibitors of RNA-dependent RNA viral replication and are useful for the treatment of RNA-dependent RNA viral infection. They are particularly useful as inhibitors of hepatitis C virus (HCV) NS5B polymerase, as inhibitors of HCV replication, and for the treatment of hepatitis C infection.

### BACKGROUND OF THE INVENTION

Hepatitis C virus (HCV) infection is a major health problem that leads to chronic liver disease, such as cirrhosis and hepatocellular carcinoma, in a substantial number of infected individuals, estimated to be 2-15% of the world's population. There are an estimated 4.5 million infected people in the United States alone, according to the U.S. Center for Disease Control. According to the World Health Organization, there are more than 200 million infected individuals worldwide, with at least 3 to 4 million people being infected each year. Once infected, about 20% of people clear the virus, but the rest harbor HCV the rest of their lives. Ten to twenty percent of chronically infected individuals eventually develop liver-destroying cirrhosis or cancer. The viral disease is transmitted parenterally by contaminated blood and blood products, contaminated needles, or sexually and vertically from infected mothers or carrier mothers to their off-spring. Current treatments for HCV infection, which are restricted to immunotherapy with recombinant interferon-α alone or in combination with the nucleoside analog ribavirin, are of limited clinical benefit. Moreover, there is no established vaccine for HCV. Consequently, there is an urgent need for improved therapeutic agents that effectively combat chronic HCV infection. The state of the art in the treatment of HCV infection has been reviewed, and reference is made to the following publications: B. Dymock, et al., “Novel approaches to the treatment of hepatitis C virus infection,” Antiviral Chemistry & Chemotherapy, 11: 79-96 (2000); H. Rosen, et al., “Hepatitis C virus: current understanding and prospects for future therapies,” Molecular Medicine Today, 5: 393-399 (1999); D. Moradpour, et al., “Current and evolving therapies for hepatitis C,” European J. Gastroenterol. Hepatol., 11: 1189-1202 (1999); R. Bartenschlager, “Candidate Targets for Hepatitis C Virus-Specific Antiviral Therapy,” Intervirology, 40: 378-393 (1997); G. M. Lauer and B. D. Walker, “Hepatitis C Virus Infection,” N. Engl. J. Med., 345: 41-52 (2001); B. W. Dymock, “Emerging therapies for hepatitis C virus infection,” Emerging Drugs, 6: 13-42 (2001); and C. Crabb, “Hard-Won Advances Spark Excitement about Hepatitis C,” Science: 506-507 (2001); the contents of all of which are incorporated by reference herein in their entirety.

Different approaches to HCV therapy have been taken, which include the inhibition of viral serine proteinase (NS3 protease), helicase, and RNA-dependent RNA polymerase (NS5B), and the development of a vaccine.

The HCV virion is an enveloped positive-strand RNA virus with a single oligoribonucleotide genomic sequence of about 9600 bases which encodes a polyprotein of about 3,010 amino acids. The protein products of the HCV gene consist of the structural proteins C, E1, and E2, and the non-structural proteins NS2, NS3, NS4A and NS4B, and NS5A and NS5B. The nonstructural (NS) proteins are believed to provide the catalytic machinery for viral replication. The NS3 protease releases NS5B, the RNA-dependent RNA polymerase from the polyprotein chain. HCV NS5B polymerase is required for the synthesis of a double-stranded RNA from a single-stranded viral RNA that serves as a template in the replication cycle of HCV. NS5B polymerase is therefore considered to be an essential component in the HCV replication complex [see K. Ishi, et al., “Expression of Hepatitis C Virus NS5B Protein: Characterization of Its RNA Polymerase Activity and RNA Binding,” Hepatology, 29: 1227-1235 (1999) and V. Lohmann, et al., “Biochemical and Kinetic Analyses of NS5B RNA-Dependent RNA Polymerase of the Hepatitis C Virus,” Virology, 249: 108-118 (1998)]. Inhibition of HCV NS5B polymerase prevents formation of the double-stranded HCV RNA and therefore constitutes an attractive approach to the development of HCV-specific antiviral therapies.

It has now been found that nucleoside compounds of the present invention and certain derivatives thereof are potent inhibitors of RNA-dependent RNA viral replication and in particular HCV replication. The 5′-triphosphate derivatives of these nucleoside compounds are inhibitors of RNA-dependent RNA viral polymerase and in particular HCV NS5B polymerase. The instant nucleoside compounds and derivatives thereof are useful to treat RNA-dependent RNA viral infection and in particular HCV infection.

It is therefore an object of the present invention to provide nucleoside compounds and certain derivatives thereof which are useful as inhibitors of RNA-dependent RNA viral polymerase and in particular as inhibitors of HCV NS5B polymerase.

It is another object of the present invention to provide nucleoside compounds and certain derivatives thereof which are useful as inhibitors of the replication of an RNA-dependent RNA virus and in particular as inhibitors of the replication of hepatitis C virus.

It is another object of the present invention to provide nucleoside compounds and certain derivatives thereof which are useful in the treatment of RNA-dependent RNA viral infection and in particular in the treatment of HCV infection.

It is another object of the present invention to provide pharmaceutical compositions comprising the nucleoside compounds of the present invention in association with a pharmaceutically acceptable carrier.

It is another object of the present invention to provide pharmaceutical compositions comprising the nucleoside compounds and derivatives thereof of the present invention for use as inhibitors of RNA-dependent RNA viral polymerase and in particular as inhibitors of HCV NS5B polymerase.

It is another object of the present invention to provide pharmaceutical compositions comprising the nucleoside compounds and derivatives thereof of the present invention for use as inhibitors of RNA-dependent RNA viral replication and in particular as inhibitors of HCV replication.

It is another object of the present invention to provide pharmaceutical compositions comprising the nucleoside compounds and derivatives thereof of the present invention for use in the treatment of RNA-dependent RNA viral infection and in particular in the treatment of HCV infection.

It is another object of the present invention to provide pharmaceutical compositions comprising the nucleoside compounds and derivatives thereof of the present invention in combination with other agents active against an RNA-dependent RNA virus and in particular against HCV.

It is another object of the present invention to provide methods for the inhibition of RNA-dependent RNA viral polymerase and in particular for the inhibition of HCV NS5B polymerase.

It is another object of the present invention to provide methods for the inhibition of RNA-dependent RNA viral replication and in particular for the inhibition of HCV replication.

It is another object of the present invention to provide methods for the treatment of RNA-dependent RNA viral infection and in particular for the treatment of HCV infection.

It is another object of the present invention to provide methods for the treatment of RNA-dependent RNA viral infection in combination with other agents active against RNA-dependent RNA virus and in particular for the treatment of HCV infection in combination with other agents active against HCV.

It is another object of the present invention to provide nucleoside compounds and certain derivatives thereof and their pharmaceutical compositions for use as a medicament for the inhibition of RNA-dependent RNA viral replication and/or the treatment of RNA-dependent RNA viral infection and in particular for the inhibition of HCV replication and/or the treatment of HCV infection.

It is another object of the present invention to provide for the use of the nucleoside compounds and certain derivatives thereof of the present invention and their pharmaceutical compositions for the manufacture of a medicament for the inhibition of RNA-dependent RNA viral replication and/or the treatment of RNA-dependent RNA viral infection and in particular for the inhibition of HCV replication and/or the treatment of HCV infection.

These and other objects will become readily apparent from the detailed description which follows.

### SUMMARY OF THE INVENTION

The present invention provides a method for inhibiting RNA-dependent RNA viral polymerase, a method for inhibiting RNA-dependent RNA viral replication, and/or a method for treating RNA-dependent viral infection in a mammal in need thereof, comprising administering to the mammal a therapeutically effective amount of a compound of structural formula I which is of the stereochemical configuration:

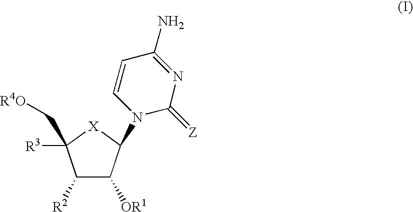

or a pharmaceutically acceptable salt thereof; wherein

X is O or S;

Z is O or S;

R¹ is hydrogen, methyl, C₁₋₁₆ alkylcarbonyl, C₂₋₁₈ alkenylcarbonyl, C₁₋₁₀ alkyloxycarbonyl, C₃₋₆ cycloalkylcarbonyl, C₃₋₆ cycloalkyloxycarbonyl, CH₂O(C═O)C₁₋₄ alkyl, CH(C₁₋₄ alkyl)O(C═O)C₁₋₄ alkyl, or an amino acyl residue of structural formula

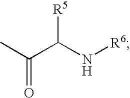

R² is hydrogen, hydroxy, fluoro, amino, methyl, methoxy, C₁₋₁₆ alkylcarbonyloxy, C₂₋₁₈ alkenylcarbonyloxy, C₁₋₁₀ alkyloxycarbonyloxy, C₃₋₆ cycloalkylcarbonyloxy, C₃₋₆ cycloalkyloxycarbonyloxy, —OCH₂O(C═O)C₁₋₄ alkyl, —OCH(C₁₋₄ alkyl)O(C═O)C₁₋₄ alkyl, or an amino acyloxy residue of structural formula

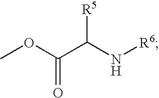

R³ is selected from the group consisting of methyl, ethynyl, cyano, aminocarbonyl, fluoromethyl, bromomethyl, trifluoromethyl, aminomethyl, fluoro, chloro, and bromo;

R⁴ is hydrogen, C₁₋₁₀ alkylcarbonyl, C₂₋₁₈ alkenylcarbonyl, C₁₋₁₀ alkyloxycarbonyl, C₃₋₆ cycloalkylcarbonyl, C₃₋₆ cycloalkyloxycarbonyl, CH₂O(C═O)C₁₋₄ alkyl, CH(C₁₋₄ alkyl)O(C═O)C₁₋₄ alkyl, P₃O₉H₄, P₂O₆H₃, P(O)R⁷R⁸, or an amino acyl residue of structural formula

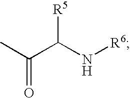

R⁵ is hydrogen, C₁₋₄ alkyl, or phenyl C₀₋₂ alkyl;

R⁶ is hydrogen, C₁₋₄ alkyl, C₁₋₄ acyl, benzoyl, C₁₋₄ alkyloxycarbonyl, phenyl C₀₋₂ alkyloxycarbonyl, C₁₋₄ alkylaminocarbonyl, phenyl C₀₋₂ alkylaminocarbonyl, C₁₋₄ alkylsulfonyl, or phenyl C₀₋₂ alkylsulfonyl; and

R⁷ and R⁸ are each independently hydroxy, OCH₂CH₂SC(═O)C₁₋₄ alkyl, OCH₂O(C═O)OC₁₋₄ alkyl, NHCHMeCO₂Me, OCH(C₁₋₄ alkyl)O(C═O)C₁₋₄ alkyl,

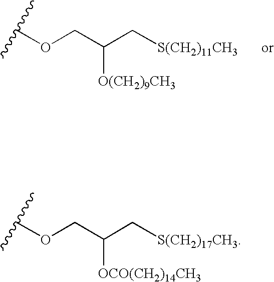

The present invention further provides novel compounds of structural formula I of the indicated stereochemical configuration which are useful as inhibitors of RNA-dependent RNA viral polymerase and in particular of HCV NS5B polymerase:

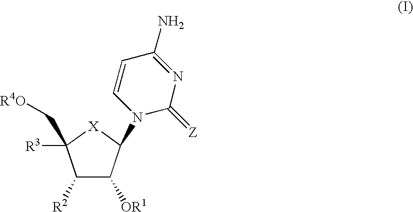

or a pharmaceutically acceptable salt thereof; wherein

X is O or S;

Z is O or S;

R¹ is hydrogen, methyl, C₁₋₁₆ alkylcarbonyl, C₂₋₁₈ alkenylcarbonyl, C₁₋₁₀ alkyloxycarbonyl, C₃₋₆ cycloalkylcarbonyl, C₃₋₆ cycloalkyloxycarbonyl, CH₂O(C═O)C₁₋₄ alkyl, CH(C₁₋₄ alkyl)O(C═O)C₁₋₄ alkyl, or an amino acyl residue of structural formula

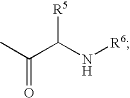

R² is hydrogen, hydroxy, fluoro, amino, methyl, methoxy, C₁₋₁₆ alkylcarbonyloxy, C₂₋₁₈ alkenylcarbonyloxy, C₁₋₁₀ alkyloxycarbonyloxy, C₃₋₆ cycloalkylcarbonyloxy, C₃₋₆ cycloalkyloxycarbonyloxy, —OCH₂O(C═O)C₁₋₄ alkyl, —OCH(C₁₋₄ alkyl)O(C═O)C₁₋₄ alkyl, or an amino acyloxy residue of structural formula

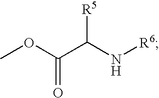

R³ is selected from the group consisting of methyl, ethynyl, cyano, aminocarbonyl, fluoromethyl, bromomethyl, trifluoromethyl, aminomethyl, fluoro, chloro, and bromo;

R⁴ is hydrogen, C₁₋₁₀ alkylcarbonyl, C₂₋₁₈ alkenylcarbonyl, C₁₋₁₀ alkyloxycarbonyl, C₃₋₆ cycloalkylcarbonyl, C₃₋₆ cycloalkyloxycarbonyl, CH₂O(C═O)C₁₋₄ alkyl, CH(C₁₋₄ alkyl)O(C═O)C₁₋₄ alkyl, P₃O₉H₄, P₂O₆H₃, P(O)R⁷R⁸, or an amino acyl residue of structural formula

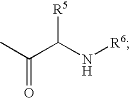

R⁵ is hydrogen, C₁₋₄ alkyl, or phenyl C₀₋₂ alkyl;

R⁶ is hydrogen, C₁₋₄ alkyl, C₁₋₄ acyl, benzoyl, C₁₋₄ alkyloxycarbonyl, phenyl C₀₋₂ alkyloxycarbonyl, C₁₋₄ alkylaminocarbonyl, phenyl C₀₋₂ alkylaminocarbonyl, C₁₋₄ alkylsulfonyl, or phenyl C₀₋₂ alkylsulfonyl; and

R⁷ and R⁸ are each independently hydroxy, OCH₂CH₂SC(=O)C₁₋₄ alkyl, OCH₂O(C═O)OC₁₋₄ alkyl, NHCHMeCO₂Me, OCH(C₁₋₄ alkyl)O(C═O)C₁₋₄ alkyl,

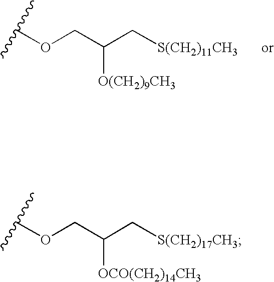

with the proviso that when X and Z are both O , R¹ and R⁴ are hydrogen, and R² is hydroxy, then R³ is not methyl, fluoromethyl, ethynyl, or cyano.

The compounds of formula I are useful as inhibitors of RNA-dependent RNA viral polymerase and in particular of HCV NS5B polymerase. They are also inhibitors of RNA-dependent RNA viral replication and in particular of HCV replication and are useful for the treatment of RNA-dependent RNA viral infection and in particular for the treatment of HCV infection.

Also encompassed within the present invention are pharmaceutical compositions containing the compounds alone or in combination with other agents active against RNA-dependent RNA virus and in particular against HCV as well as methods for the inhibition of RNA-dependent RNA viral replication and for the treatment of RNA-dependent RNA viral infection.

### DETAILED DESCRIPTION OF THE INVENTION

The present invention provides a method for inhibiting RNA-dependent RNA viral polymerase, a method for inhibiting RNA-dependent RNA viral replication, and/or a method for treating RNA-dependent viral infection in a mammal in need thereof, comprising administering to the mammal a therapeutically effective amount of a compound of structural formula I which is of the stereochemical configuration:

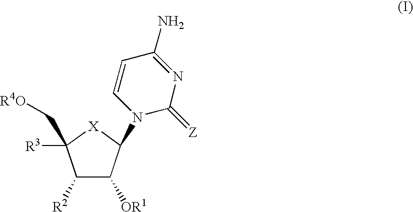

or a pharmaceutically acceptable salt thereof; wherein

X is O or S;

Z is O or S;

R¹ is hydrogen, methyl, C₁₋₁₆ alkylcarbonyl, C₂₋₁₈ alkenylcarbonyl, C₁₋₁₀ alkyloxycarbonyl, C₃₋₆ cycloalkylcarbonyl, C₃₋₆ cycloalkyloxycarbonyl, CH₂O(C═O)C₁₋₄ alkyl, CH(C₁₋₄ alkyl)O(C═O)C₁₋₄ alkyl, or an amino acyl residue of structural formula

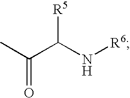

R² is hydrogen, hydroxy, fluoro, amino, methyl, methoxy, C₁₋₁₆ alkylcarbonyloxy, C₂₋₁₈ alkenylcarbonyloxy, C₁₋₁₀ alkyloxycarbonyloxy, C₃₋₆ cycloalkylcarbonyloxy, C₃₋₆ cycloalkyloxycarbonyloxy, —OCH₂O(C═O)C₁₋₄ alkyl, —OCH(C₁₋₄ alkyl)O(C═O)C₁₋₄ alkyl, or an amino acyloxy residue of structural formula

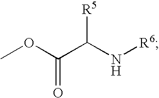

R³ is selected from the group consisting of methyl, ethynyl, cyano, aminocarbonyl, fluoromethyl, bromomethyl, trifluoromethyl, aminomethyl, fluoro, chloro, and bromo;

R⁴ is hydrogen, C₁₋₁₀ alkylcarbonyl, C₂₋₁₈ alkenylcarbonyl, C₁₋₁₀ alkyloxycarbonyl, C₃₋₆ cycloalkylcarbonyl, C₃₋₆ cycloalkyloxycarbonyl, CH₂O(C═O)C₁₋₄ alkyl, CH(C₁₋₄ alkyl)O(C═O)C₁₋₄ alkyl, P₃O₉H₄, P₂O₆H₃, P(O)R⁷R⁸, or an amino acyl residue of structural formula

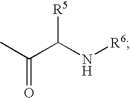

R⁵ is hydrogen, C₁₋₄ alkyl, or phenyl C₀₋₂ alkyl;

R⁶ is hydrogen, C₁₋₄ alkyl, C₁₋₄ acyl, benzoyl, C₁₋₄ alkyloxycarbonyl, phenyl C₀₋₂ alkyloxycarbonyl, C₁₋₄ alkylaminocarbonyl, phenyl C₀₋₂ alkylaminocarbonyl, C₁₋₄ alkylsulfonyl, or phenyl C₀₋₂ alkylsulfonyl; and

R⁷ and R⁸ are each independently hydroxy, OCH₂CH₂SC(═O)C₁₋₄ alkyl, OCH₂O(C═O)OC₁₋₄ alkyl, NHCHMeCO₂Me, OCH(C₁₋₄ alkyl)O(C═O)C₁₋₄ alkyl,

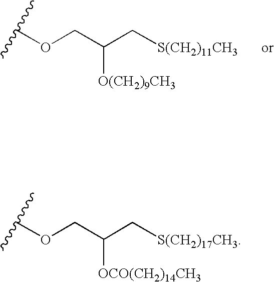

In one embodiment of the present invention is the method of inhibiting RNA-dependent RNA viral polymerase, inhibiting RNA-dependent viral replication, and/or treating RNA-dependent RNA viral infection with a compound of structural formula I wherein X and Z are O. In a class of this embodiment, R¹ is hydrogen, methyl, C₁₋₁₆ alkylcarbonyl, or an aminoacyl residue of structural formula

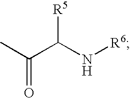

R² is hydroxy, C₁₋₆ alkylcarbonyloxy, or an amino acyloxy residue of structural formula

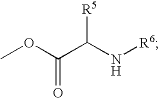

R³ is methyl or fluoromethyl;

R⁴ is hydrogen, C₁₋₁₀ alkylcarbonyl, P₃O₉H₄, or an amino acyl residue of structural formula

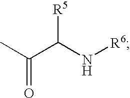

and R⁵ and R⁶ are as defined above.

In a second embodiment of the present invention, the RNA-dependent RNA viral polymerase is a positive-sense single-stranded RNA-dependent RNA viral polymerase. In a class of this embodiment, the positive-sense single-stranded RNA-dependent RNA viral polymerase is a Flaviviridae viral polymerase or a Picornaviridae viral polymerase. In a subclass of this class, the Picornaviridae viral polymerase is rhinovirus polymerase, poliovirus polymerase, or hepatitis A virus polymerase. In a second subclass of this class, the Flaviviridae viral polymerase is selected from the group consisting of hepatitis C virus polymerase, yellow fever virus polymerase, dengue virus polymerase, West Nile virus polymerase, Japanese encephalitis virus polymerase, Banzi virus polymerase, and bovine viral diarrhea virus (BVDV) polymerase. In a subclass of this subclass, the Flaviviridae viral polymerase is hepatitis C virus polymerase.

In a third embodiment of the present invention, the RNA-dependent RNA viral replication is a positive-sense single-stranded RNA-dependent RNA viral replication. In a class of this embodiment, the positive-sense single-stranded RNA-dependent RNA viral replication is Flaviviridae viral replication or Picornaviridae viral replication. In a subclass of this class, the Picornaviridae viral replication is rhinovirus replication, poliovirus replication, or hepatitis A virus replication. In a second subclass of this class, the Flaviviridae viral replication is selected from the group consisting of hepatitis C virus replication, yellow fever virus replication, dengue virus replication, West Nile virus replication, Japanese encephalitis virus replication, Banzi virus replication, and bovine viral diarrhea virus replication. In a subclass of this subclass, the Flaviviridae viral replication is hepatitis C virus replication.

In a fourth embodiment of the present invention, the RNA-dependent RNA viral infection is a positive-sense single-stranded RNA-dependent viral infection. In a class of this embodiment, the positive-sense single-stranded RNA-dependent RNA viral infection is Flaviviridae viral infection or Picornaviridae viral infection. In a subclass of this class, the Picornaviridae viral infection is rhinovirus infection, poliovirus infection, or hepatitis A virus infection. In a second subclass of this class, the Flaviviridae viral infection is selected from the group consisting of hepatitis C virus infection, yellow fever virus infection, dengue virus infection, West Nile virus infection, Japanese encephalitis virus infection, Banzi virus infection, and bovine viral diarrhea virus infection. In a subclass of this subclass, the Flaviviridae viral infection is hepatitis C virus infection.

Illustrative of the invention is a method for inhibiting RNA-dependent RNA viral polymerase, inhibiting RNA-dependent RNA viral replication, and/or treating RNA-dependent RNA viral infection wherein the compound is 4′-C-methylcytidine or 4′-C-(fluoromethyl)cytidine or their corresponding 5′-triphosphate derivative or a pharmaceutically acceptable salt thereof.

Another aspect of the present invention concerns novel compounds of structural formula I of the indicated stereochemical configuration:

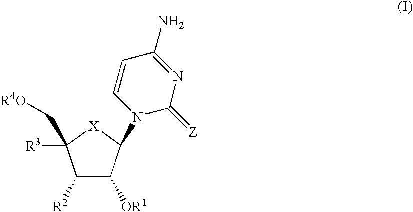

or a pharmaceutically acceptable salt thereof; wherein

X is O or S;

Z is O or S;

R¹ is hydrogen, methyl, C₁₋₁₆ alkylcarbonyl, C₂₋₁₈ alkenylcarbonyl, C₁₋₀ alkyloxycarbonyl, C₃₋₆ cycloalkylcarbonyl, C₃₋₆ cycloalkyloxycarbonyl, CH₂O(C═O)C₁₋₄ alkyl, CH(C₁₋₄ alkyl)O(C═O)C₁₋₄ alkyl, or an amino acyl residue of structural formula

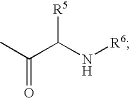

R² is hydrogen, hydroxy, fluoro, amino, methyl, methoxy, C₁₋₁₆ alkylcarbonyloxy, C₂₋₁₈ alkenylcarbonyloxy, C₁₋₁₀ alkyloxycarbonyloxy, C₃₋₆ cycloalkylcarbonyloxy, C₃₋₆ cycloalkyloxycarbonyloxy, —OCH₂O(C═O)C₁₋₄ alkyl, —OCH(C₁₋₄ alkyl)O(C═O)C₁₋₄ alkyl, or an amino acyloxy residue of structural formula

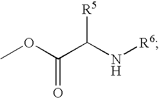

R³ is selected from the group consisting of methyl, ethynyl, cyano, aminocarbonyl, fluoromethyl, bromomethyl, trifluoromethyl, aminomethyl, fluoro, chloro, and bromo;

R⁴ is hydrogen, C₁₋₁₀ alkylcarbonyl, C₂₋₁₈ alkenylcarbonyl, C₁₋₀ alkyloxycarbonyl, C₃₋₆ cycloalkylcarbonyl, C₃₋₆ cycloalkyloxycarbonyl, CH₂O(C═O)C₁₋₄ alkyl, CH(C₁₋₄ halkyl)O(C═O)C₁₋₄ alkyl, P₃O₉H₄, P₂O₆H₃, P(O)R⁷R⁸, or an amino acyl residue of structural formula

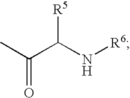

R⁵ is hydrogen, C₁₋₄ alkyl, or phenyl C₀₋₂ alkyl;

R⁶ is hydrogen, C₁₋₄ alkyl, C₁₋₄ acyl, benzoyl, C₁₋₄ alkyloxycarbonyl, phenyl C₀₋₂ alkyloxycarbonyl, C₁₋₄ alkylaminocarbonyl, phenyl C₀₋₂ alkylaminocarbonyl, C₁₋₄ alkylsulfonyl, or phenyl C₀₋₂ alkylsulfonyl; and

R⁷ and R⁸ are each independently hydroxy, OCH₂CH₂SC(═O)C₁₋₄ alkyl, OCH₂O(C═O)OC₁₋₄ alkyl, NHCHMeCO₂Me, OCH(C₁₋₄ alkyl)O(C═O)C₁₋₄ alkyl,

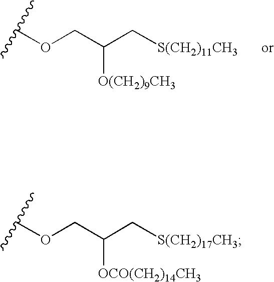

with the proviso that when X and Z are both O, R¹ and R⁴ are hydrogen, and R² is hydroxy, then

R³ is not methyl, fluoromethyl, ethynyl, or cyano.

In one embodiment of the compounds of structural formula I, X and Z are O. In a class of this embodiment, R¹ is hydrogen, C¹⁻¹⁶ alkylcarbonyl, or an aminoacyl residue of structural formula

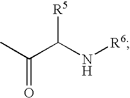

R² is hydroxy, C₁₋₁₆ alkylcarbonyloxy, or an amino acyloxy residue of structural formula

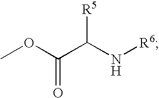

R³ is methyl or fluoromethyl;

R⁴ is hydrogen, C₁₋₁₀ alkylcarbonyl, P₃O₉H₄, or an amino acyl residue of structural formula

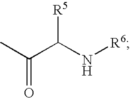

and R⁵ and R⁶ are as defined above;

with the proviso that when R¹ and R⁴ are hydrogen, and R² is hydroxy, then R³ is not methyl, fluoromethyl, ethynyl, or cyano.

The nucleoside compounds of the present invention are useful as inhibitors of positive-sense single-stranded RNA-dependent RNA viral polymerase, inhibitors of positive-sense single-stranded RNA-dependent RNA viral replication, and/or for the treatment of positive-sense single-stranded RNA-dependent RNA viral infection. In a class of this embodiment, the positive-sense single-stranded RNA-dependent RNA virus is a Flaviviridae virus or a Picornaviridae virus. In a subclass of this class, the Picornaviridae virus is a rhinovirus, a poliovirus, or a hepatitis A virus. In a second subclass of this class, the Flaviviridae virus is selected from the group consisting of hepatitis C virus, yellow fever virus, dengue virus, West Nile virus, Japanese encephalitis virus, Banzi virus, and bovine viral diarrhea virus (BVDV). In a subclass of this subclass, the Flaviviridae virus is hepatitis C virus.

Throughout the instant application, the following terms have the indicated meanings:

The alkyl groups specified above are intended to include those alkyl groups of the designated length in either a straight or branched configuration. Exemplary of such alkyl groups are methyl, ethyl, propyl, isopropyl, butyl, sec-butyl, tertiary butyl, pentyl, isopentyl, hexyl, isohexyl, and the like.

The term “alkenyl” shall mean straight or branched chain alkenes of two to six total carbon atoms, or any number within this range (e.g., ethenyl, propenyl, butenyl, pentenyl, etc.).

The term “alkynyl” shall mean straight or branched chain alkynes of two to six total carbon atoms, or any number within this range (e.g., ethynyl, propynyl, butynyl, pentynyl, etc.).

The term “cycloalkyl” shall mean cyclic rings of alkanes of three to eight total carbon atoms, or any number within this range (i.e., cyclopropyl, cyclobutyl, cyclopentyl, cyclohexyl, cycloheptyl, or cyclooctyl).

The term “alkoxy” refers to straight or branched chain alkoxides of the number of carbon atoms specified (e.g., C₁₋₄ alkoxy), or any number within this range [i.e., methoxy (MeO—), ethoxy, isopropoxy, etc.].

The term “alkylthio” refers to straight or branched chain alkylsulfides of the number of carbon atoms specified (e.g., C₁₋₄ alkylthio), or any number within this range [i.e., methylthio (MeS—), ethylthio, isopropylthio, etc.].

The term “alkylamino” refers to straight or branched alkylamines of the number of carbon atoms specified (e.g., C₁₋₄ alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C₁₋₆ alkylsulfonyl), or any number within this range [i.e., methylsulfonyl (MeSO₂—), ethylsulfonyl, isopropylsulfonyl, etc.].

The term “alkyloxycarbonyl” refers to straight or branched chain esters of a carboxylic acid derivative of the present invention of the number of carbon atoms specified (e.g., C₁₋₄ alkyloxycarbonyl), or any number within this range [i.e., methyloxycarbonyl (MeOCO—), ethyloxycarbonyl, or butyloxycarbonyl].

The term “halogen” is intended to include the halogen atoms fluorine, chlorine, bromine and iodine.

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally.

When R⁵ in the amino acyl or amino acyloxy residue shown below is not hydrogen,

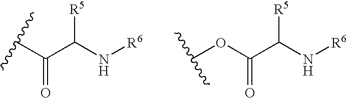

the amino acyl or amino acyloxy residue contains an asymmetric center and is intended to include the individual R- and S-enantioners as well as RS-racemic mixtures.

The term “5′-triphosphate” refers to a triphosphoric acid ester derivative of the 5′-hydroxyl group of a nucleoside compound of the present invention having the following general structural formula II:

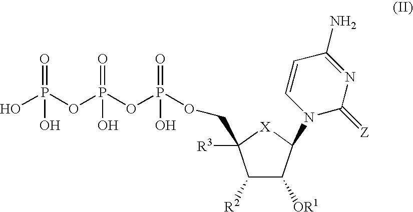

wherein X, Z, and R¹-R³ are as defined above.

The compounds of the present invention are also intended to include pharmaceutically acceptable salts of the triphosphate ester as well as pharmaceutically acceptable salts of 5′-monophosphate and 5′-diphosphate ester derivatives of the structural formulae III and IV, respectively,

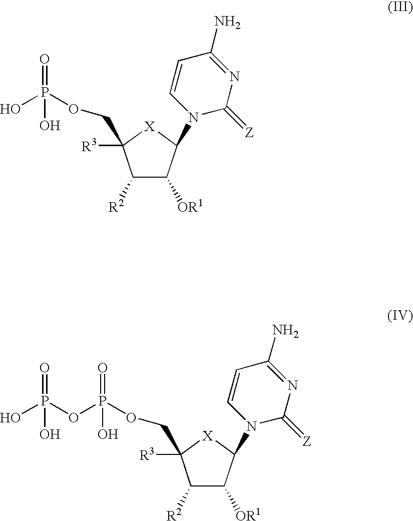

The term “composition”, as in “pharmaceutical composition,” is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

The terms “administration of” and “administering a” compound should be understood to mean providing a compound of the invention or a prodrug of a compound of the invention to the subject in need.

The subject treated in the present methods is generally a mammal, preferably a human being, male or female. The term “therapeutically effective amount” means the amount of the subject compound that will elicit the biological or medical response of a tissue, system, animal or human that is being sought by the researcher, veterinarian, medical doctor or other clinician.

Another aspect of the present invention is concerned with a method of inhibiting HCV NS5B polymerase, inhibiting HCV replication, or treating HCV infection with a compound of the present invention in combination with one or more agents useful for treating HCV infection. Such agents active against HCV include, but are not limited to, ribavirin, levovirin, viramidine, thymosin alpha-1, interferon-β, intererfon-α, pegylated interferon-α (peginterferon-α), a combination of intererfon-α and ribavirin, a combination of pegintererfon-α and ribavirin, a combination of intererfon-α and levovirin, and a combination of pegintererfon-α and levovirin. Interferon-α includes, but is not limited to, recombinant interferon-α2a (such as Roferon interferon available from Hoffmann-LaRoche, Nutley, N.J.), pegylated intererfon-α2a (Pegasys™), intererfon-α2b (such as Intron-A interferon available from Schering Corp., Kenilworth, N.J.), pegylated intererfon-α2b (PegIntron™), a recombinant consensus interferon (such as interferon alphacon-1), and a purified intererfon-α product. Amgen's recombinant consensus interferon has the brand name Infergen®. Levovirin is the L-enantiomer of ribavirin which has shown immunomodulatory activity similar to ribavirin. Viramidine represents an analog of ribavirin disclosed in WO 01/60379 (assigned to ICN Pharmaceuticals). In accordance with this method of the present invention, the individual components of the combination can be administered separately at different times during the course of therapy or concurrently in divided or single combination forms. The instant invention is therefore to be understood as embracing all such regimes of simultaneous or alternating treatment, and the term “administering” is to be interpreted accordingly. It will be understood that the scope of combinations of the compounds of this invention with other agents useful for treating HCV infection includes in principle any combination with any pharmaceutical composition for treating HCV infection. When a compound of the present invention or a pharmaceutically acceptable salt thereof is used in combination with a second therapeutic agent active against HCV, the dose of each compound may be either the same as or different from the dose when the compound is used alone.

For the treatment of HCV infection, the compounds of the present invention may also be administered in combination with an agent that is an inhibitor of HCV NS3 serine protease. HCV NS3 serine protease is an essential viral enzyme and has been described to be an excellent target for inhibition of HCV replication. Both substrate and non-substrate based inhibitors of HCV NS3 protease inhibitors are disclosed in WO 98/22496, WO 98/46630, WO 99/07733, WO 99/07734, WO 99/38888, WO 99/50230, WO 99/64442, WO 00/09543, WO 00/59929, GB-2337262, WO 02/48116, WO 02/48172, and U.S. Pat. No. 6,323,180. HCV NS3 protease as a target for the development of inhibitors of HCV replication and for the treatment of HCV infection is discussed in B. W. Dymock, “Emerging therapies for hepatitis C virus infection,” Emerging Drugs, 6: 13-42 (2001).

Ribavirin, levovirin, and viramidine may exert their anti-HCV effects by modulating intracellular pools of guanine nucleotides via inhibition of the intracellular enzyme inosine monophosphate dehydrogenase (IMPDH). IMPDH is the rate-limiting enzyme on the biosynthetic route in de novo guanine nucleotide biosynthesis. Ribavirin is readily phosphorylated intracellularly and the monophosphate derivative is an inhibitor of IMPDH. Thus, inhibition of IMPDH represents another useful target for the discovery of inhibitors of HCV replication. Therefore, the compounds of the present invention may also be administered in combination with an inhibitor of IMPDH, such as VX-497, which is disclosed in WO 97/41211 and WO 01/00622 (assigned to Vertex); another IMPDH inhibitor, such as that disclosed in WO 00/25780 (assigned to Bristol-Myers Squibb); or mycophenolate mofetil [see A. C. Allison and E. M. Eugui, Agents Action, 44 (Suppl.): 165 (1993)].

For the treatment of HCV infection, the compounds of the present invention may also be administered in combination with the antiviral agent amantadine (1-aminoadamantane) [for a comprehensive description of this agent, see J. Kirschbaum, Anal. Profiles Drug Subs. 12: 1-36 (1983)].

The compounds of the present invention may also be combined for the treatment of HCV infection with antiviral 1′-C, 2′-C-, or 3′-C-branched ribonucleosides disclosed in R. E. Harry-O'kuru, et al., J. Org. Chem., 62: 1754-1759 (1997); M. S. Wolfe, et al., Tetrahedron Lett., 36: 7611-7614 (1995); U.S. Pat. No. 3,480,613 (Nov. 25, 1969); International Publication Number WO 01/90121 (29 Nov. 2001); International Publication Number WO 01/92282 (6 Dec. 2001); and International Publication Number WO 02/32920 (25 Apr. 2002); the contents of each of which are incorporated by reference in their entirety. Such branched ribonucleosides include, but are not limited to, 2′-C-methylcytidine, 2′-C-methyluridine, 2′-C-methyladenosine, 2′-C-methylguanosine, and 9-(2-C-methyl-β-D-ribofuranosyl)-2,6-diaminopurine.

The compounds of the present invention may also be combined for the treatment of HCV infection with other nucleosides having anti-HCV properties, such as those disclosed in WO 02/51425 (4 Jul. 2002), assigned to Mitsubishi Pharma Corp.; WO 01/79246, WO 02/32920 (25 Apr. 2002), and WO 02/48165 (20 Jun. 2002), assigned to Pharmasset, Ltd.; WO 01/68663 (20 Sep. 2001), assigned to ICN Pharmaceuticals; WO 99/43691 (2 Sep. 1999); WO 02/18404 (7 Mar. 2002), assigned to Hoffmann-LaRoche; U.S. 2002/0019363 (14 Feb. 2002); WO 02/057287 (25 Jul. 2002), assigned to Merck & Co. and Isis Pharmaceuticals; and WO 02/057425 (25 Jul. 2002), assigned to Merck & Co. and Isis Pharmaceuticals.

The compounds of the present invention may also be combined for the treatment of HCV infection with non-nucleoside inhibitors of HCV polymerase such as those disclosed in WO 01/77091 (18 Oct. 2001), assigned to Tularik, Inc.; WO 01/47883 (5 Jul. 2001), assigned to Japan Tobacco, Inc.; WO 02/04425 (17 Jan. 2002), assigned to Boehringer Ingelheim; WO 02/06246 (24 Jan. 2002), assigned to Istituto di Ricerche di Biologia Moleculare P. Angeletti S. P. A.; and WO 02/20497 (3 Mar. 2002).

By “pharmaceutically acceptable” is meant that the carrier, diluent, or excipient must be compatible with the other ingredients of the formulation and not deleterious to the recipient thereof.

Also included within the present invention are pharmaceutical compositions comprising the nucleoside compounds and derivatives thereof of the present invention in association with a pharmaceutically acceptable carrier. Another example of the invention is a pharmaceutical composition made by combining any of the compounds described above and a pharmaceutically acceptable carrier. Another illustration of the invention is a process for making a pharmaceutical composition comprising combining any of the compounds described above and a pharmaceutically acceptable carrier.

Also included within the present invention are pharmaceutical compositions useful for inhibiting RNA-dependent RNA viral polymerase in particular HCV NS5B polymerase comprising an effective amount of a compound of the present invention and a pharmaceutically acceptable carrier. Pharmaceutical compositions useful for treating RNA-dependent RNA viral infection in particular HCV infection are also encompassed by the present invention as well as a method of inhibiting RNA-dependent RNA viral polymerase in particular HCV NS5B polymerase and a method of treating RNA-dependent viral replication and in particular HCV replication. Additionally, the present invention is directed to a pharmaceutical composition comprising a therapeutically effective amount of a compound of the present invention in combination with a therapeutically effective amount of another agent active against RNA-dependent RNA virus and in particular against HCV. Agents active against HCV include, but are not limited to, ribavirin, levovirin, viramidine, thymosin alpha-1, an inhibitor of HCV NS3 serine protease, intererfon-α, pegylated intererfon-α (pegintererfon-α), a combination of intererfon-α and ribavirin, a combination of pegintererfon-α and ribavirin, a combination of intererfon-α and levovirin, and a combination of pegintererfon-α and levovirin. Interferon-α includes, but is not limited to, recombinant intererfon-α2a (such as Roferon interferon available from Hoffmann-LaRoche, Nutley, N.J.), intererfon-α2b (such as Intron-A interferon available from Schering Corp., Kenilworth, N.J.), a consensus interferon, and a purified interferon-α product. For a discussion of ribavirin and its activity against HCV, see J. O. Saunders and S. A. Raybuck, “Inosine Monophosphate Dehydrogenase: Consideration of Structure, Kinetics, and Therapeutic Potential,” Ann. Rep. Med. Chem., 35: 201-210 (2000).

Another aspect of the present invention provides for the use of the nucleoside compounds and derivatives thereof and their pharmaceutical compositions for the manufacture of a medicament for the inhibition of RNA-dependent RNA viral replication, in particular HCV replication, and/or the treatment of RNA-dependent RNA viral infection, in particular HCV infection. Yet a further aspect of the present invention provides for the nucleoside compounds and derivatives thereof and their pharmaceutical compositions for use as a medicament for the inhibition of RNA-dependent RNA viral replication, in particular HCV replication, and/or for the treatment of RNA-dependent RNA viral infection, in particular HCV infection.

The pharmaceutical compositions of the present invention comprise a compound of structural formula I as an active ingredient or a pharmaceutically acceptable salt thereof, and may also contain a pharmaceutically acceptable carrier and optionally other therapeutic ingredients.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of structural formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of structural formula I may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

Any suitable route of administration may be employed for providing a mammal, especially a human with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of structural formula I are administered orally.

For oral administration to humans, the dosage range is 0.01 to 1000 mg/kg body weight in divided doses. In one embodiment the dosage range is 0.1 to 100 mg/kg body weight in divided doses. In another embodiment the dosage range is 0.5 to 20 mg/kg body weight in divided doses. For oral administration, the compositions are preferably provided in the form of tablets or capsules containing 1.0 to 1000 milligrams of the active ingredient, particularly, 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated.

The effective dosage of active ingredient employed may vary depending on the particular compound employed, the mode of administration, the condition being treated and the severity of the condition being treated. Such dosage may be ascertained readily by a person skilled in the art. This dosage regimen may be adjusted to provide the optimal therapeutic response.

The compounds of the present invention contain one or more asymmetric centers and can thus occur as racemates and racemic mixtures, single enantiomers, diastereomeric mixtures and individual diastereomers. The present invention is meant to comprehend nucleoside compounds having the β-D stereochemical configuration for the five-membered furanose ring as depicted in the structural formula below, that is, nucleoside compounds in which C-1 nucleobase and the C-4 hydroxymethyl substituents of the five-membered furanose ring have the β-stereochemical configuration (“up” orientation as denoted by a bold line).

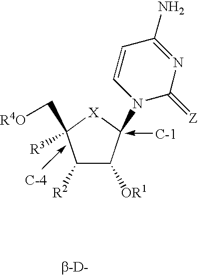

Some of the compounds described herein contain olefinic double bonds, and unless specified otherwise, are meant to include both E and Z geometric isomers.

Some of the compounds described herein may exist as tautomers such as keto-enol tautomers. The individual tautomers as well as mixtures thereof are encompassed with compounds of structural formula I.

Compounds of structural formula I may be separated into their individual diastereoisomers by, for example, fractional crystallization from a suitable solvent, for example methanol or ethyl acetate or a mixture thereof, or via chiral chromatography using an optically active stationary phase.

Alternatively, any stereoisomer of a compound of the structural formula I may be obtained by stereospecific synthesis using optically pure starting materials or reagents of known configuration.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as acetate or maleate, can be employed. Included are those esters and acyl groups known in the art for modifying the solubility or hydrolysis characteristics for use as sustained-release or prodrug formulations.

### Preparation of the Nucleoside Compounds and Derivatives of the Invention

The nucleoside compounds and derivatives thereof of the present invention can be prepared following synthetic methodologies well-established in the practice of nucleoside and nucleotide chemistry. Reference is made to the following text for a description of synthetic methods used in the preparation of the compounds of the present invention: “Chemistry of Nucleosides and Nucleotides,” L. B. Townsend, ed., Vols. 1-3, Plenum Press, 1988, which is incorporated by reference herein in its entirety. Reference is also made to the following article and references cited therein for methods of preparation of nucleosides wherein the ring oxygen is replaced with a sulfur: L. Bellon, et al., “4′-Thio-RNA: a novel class of sugar-modified β-RNA,” ACS Symposium Series (1994), 580 (Carbohydrate Modifications in Antisense Research), pages 68-79.

Compounds wherein R³ is fluoro may be prepared following procedures detailed in J. Org. Chem., 41: 3010-3017 (1976) for the preparation of 4′-fluorouridine. The 4′-fluorouridines may be converted into the 4′-fluorocytidines following the method described in J. Chem. Soc. Perkin Trans. 1, 1171-1176 (1982).

The examples below provide citations to literature publications which contain details for the preparation of intermediates employed in the preparation of final compounds of the present invention. The nucleoside compounds of the present invention were prepared according to procedures detailed in the following examples. The examples are not intended to be limitations on the scope of the instant invention in any way, and they should not be so construed. Those skilled in the art of nucleoside and nucleotide synthesis will readily appreciate that known variations of the conditions and processes of the following preparative procedures can be used to prepare these and other compounds of the present invention. All temperatures are degrees Celsius unless otherwise noted.

### EXAMPLE 1

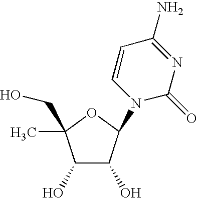

### 4′-C-Methylcytidine

Step A: 1,2:5,6-di-O-isopropylidene-α-D-ribo-hexofuranos-3-ulose

Water (640 mL), potassium metaperiodate (211 g, 0.92 mol), potassium carbonate (23 g), and ruthenium dioxide hydrate reagent (2.56 g) were respectively added to a solution of 1,2:5,6-di-O-isopropylidene-α-D-glucofuranose (160 g, 0.61 mol) in alcohol-free chloroform (704 mL). The reaction mixture was stirred at room temperature for 12 h. Additional portions of potassium metaperiodate (84 g), ruthenium dioxide hydrate reagent (1.0 g), and potassium carbonate (9.3 g) were added and the stirring was continued for total of 15 h at the same temperature. The reaction mixture was then quenched with isopropanol (90 mL). The solids were filtered off and the organic layer was concentrated under reduced pressure to a crude residue which was crystallized with dichloromethane-hexane to give the title compound as a white solid.

Step B: 1,2:5,6-di-O-isopropylidene-α-D-allofuranose

The title compound was prepared according to a published procedure [Carbohydr. Res., 24: (1972) 192-197] and crystallized from dichloromethane-hexane to give the title compound as white crystals.

¹H NMR (CDCl₃, 200 MHz): δ 5.82 (d, 1H, J=3.8 Hz, H-1), 4.62 (dd, 1H, 3.8 Hz, J=4.0 Hz, H-2), 4.36-4.27 (m, 1H), 4.13-3.98 (m, 3H), 3.82 (dd, 1H), 2.61 (d, 1H, J=8.4 Hz, 3-OH), 1.58 (s, 3H, CH₃), 1.47 (s, 3H, CH₃), 1.39 (s, 3H, CH₃), 1.38 (s, 3H, CH₃).

Step C: 1,2:5,6-di-O-isopropylidene-3-O-benzyl-α-D-allofuranose

Powered KOH (48 g) was added to a solution of 1,2:5,6-di-O-isopropylidene-α-D-allofuranose (43.7 g, 0.168 mol) and 18-crown-6 (5.0 g) in dry THF (200 mL). The reaction mixture was stirred at room temperature for 40-60 min. Benzyl bromide (43 g, 0.253 mol) was then added dropwise and the stirring was continued for 2-3 h at the same temperature. The solids were filtered off and the organic layer was concentrated under reduced pressure. The crude residue was dissolved in dichloromethane, washed with saturated brine solution, dried (MgSO₄) and concentrated to a crude residue, which was crystallized with hexane to give the title compound.

¹H NMR (CDCl₃, 200 MHz): δ 7.57-7.49 (m, 5H, ArH), 5.74 (d, 1H, J=3.8 Hz, H-1), 4.91 (d, 1H, J_{gem}=11.6 Hz, OCH_{A}Ph), 4.74 (d, 1H, J_{gem}=11.6 Hz, OCH_{B}Ph), 4.67 (t, 1H), 4.38-4.29(m, 1H), 4.17-4.11 (m, 1H), 3.99-3.87 (m, 3H), 1.40 (s, 3H, CH₃), 1.39 (s, 3H, CH₃), 1.37 (s, 3H, CH₃), 1.36 (s, 3H, CH₃).

Steps D-F: 1,2-O-isopropylidene-3-O-benzyl-4-C-hydroxymethyl-β-D-ribofuranose

The title compound was prepared according to literature methods in three steps [Tetrahedron Lett., 33: 37 (1992), J. Org. Chem., 44: 1301 (1979), and J. Org. Chem., 44: 1309 (1979)].

¹H NMR (CD₃OD, 200 MHz): δ 7.40-7.20 (m, 5H, ArH), 5.77 (d, 1H, J=3.8Hz, H-1), 4.76-4.70 (m, 2H, H-2, OCH_{A}Ph), 4.56 (d, 1H, J_{gem}=11.6 Hz, OCH_{B}Ph), 4.25 (d, 1H, J=5.2 Hz, H-3), 4.02 (d, 1H, J=12.2Hz, CHO), 3.71 (dd, 2H, CH₂O), 3.46 (d, 1H, CHO), 1.60 (s, 3H, CH₃), 1.35 (s, 3H, CH₃).

¹³C NMR (CD₃OD, 50 MHz): δ 138.14, 128.20, 127.69, 113.22 (ketal carbon), 104.63 (C-1), 87.47, 78.93, 78.42, 72.38, 62.73, 62.24, 25.78 (CH₃), 25.08 (CH₃).

Step G: 1,2-O-isopropylidene-3,5-di-O-benzyl-4-C-hydroxymethyl-α-D-ribofuranose

Powered KOH (5.8 g, 103.57 mmol) was added to a solution of 1,2-O-isopropylidene-3-O-benzyl-4-hydroxymethyl-α-D-ribofuranose (9.73 g, 31.39 mmol) and 18-crown-6 (963 mg) in dry THF (32 mL). The reaction mixture was stirred at 50° C. for 40-60 min. The mixture was cooled to room temperature, and benzyl bromide (5.88 g, 34.59 mmol) was added dropwise and the stirring was continued at room temperature for 2-3 h. The solids were filtered off and the organic layer was concentrated to a crude residue which was passed through a column of silica gel eluted with hexane-ethyl acetate (4:1) to give the title compound as a syrup.

¹H NMR (CD₂Cl₂, 200 MHz): δ 7.38-7.31 (m, 10H, ArH), 5.78 (d, 1H, J=3.8Hz, H-1), 4.78(d, 1H, J_{gem}=11.6Hz, OCH_{A}Ph), 4.71 (dd, 1H, H-2), 4.55-4.49 (m, 3H), 4.28 (d, 1H, J=5.4 Hz, H-3), 3.96-3.72 (m, 2H), 3.65-3.56 (dd, 2H), 2.34 (dd, 1H, OH), 1.62 (s, 3H, CH₃), 1.36 (s, 3H, CH₃).

¹³C NMR (CD₂Cl₂, 50 MHz): δ 138.55, 137.88, 128.66, 128.52, 128.18, 128.01, 127.88, 127.80, 113.63 (ketal carbon), 104.74 (C-1), 86.69, 79.21, 78.99, 73.75, 72.72, 72.04, 63.19, 26.69 (CH₃), 26.10 (CH₃).

Step H: 1,2-O-isopropylidene-3,5-di-O-benzyl-4-C-iodomethyl-α-D-ribofuranose

Iodine (14.2 g, 55.83 mmol) was added to a solution of 1,2-O-isopropylidene-3,5-di-O-benzyl-4-hydroxymethyl-α-D-ribofuranose (11.51 g, 28.78 mmol), triphenylphosphine (29.17 g, 111.22 mmol), and imidazole (7.56 g, 111.06 mmol) in a mixture of dry benzene (120 mL) and dry 1,4-dioxane (30 mL). The reaction mixture was stirred at 85° C. under N₂ atmosphere for 24 h. Additional portions of triphenylphosphine (9.72 g), imidazole (2.52 g) and iodine (4.73 g) were added and the stirring was continued for total of 36 h. The reaction mixture was concentrated into a crude residue which was poured into 10% Na₂S₂O₃ solution and extracted with ethyl acetate. The organic layer was washed with saturated aqueous NaHCO₃ solution, saturated brine solution, dried (MgSO₄) and concentrated to a crude residue, which was applied to a column of silica gel eluted with hexane-ethyl acetate (4:1) to give the title compound as a syrup.

¹H NMR (CD₂Cl₂, 200 MHz): δ 7.35-7.32 (m, 5H, ArH), 5.72 (d, 1H, J=3.8Hz, H-1), 4.75-4.54 (m, 5H, H-2, 2 OCH₂Ph), 4.28 (d, 1H, J=5.0Hz, H-3), 3.93 (d, 1H, J_{gem}=11.2 Hz, CHI), 3.55 (dd, 2H, CH₂OBn), 3.49 (d, 1H, J_{gem}=11.2Hz, CHI), 1.59 (s, 3H, CH₃), 1.33 (s, 3H, CH₃).

¹³C NMR (CD₂Cl₂, 50 MHz): δ 138.32, 137.92, 128.56, 128.53, 128.08, 128.00, 127.96, 127.87, 113.67, 109.99, 103.78, 83.93, 79.34, 74.70, 73.80, 72.79, 26.89, 25.96, 11.34.

Step I: 1,2-O-isopropylidene-3,5-di-O-benzyl-4-C-methyl-α-D-ribofuranose

A solution of 1,2-O-isopropylidene-3,5-di-O-benzyl-4-iodomethyl-α-D-ribofuranose (7.48 g, 12.99 mmol), tributyltin hydride (18.90 g, 64.95 mmol) and 2,2′-azobisisobutyronitrile (AIBN) (1.42 g, 8.64 mmol) in dry benzene (200 ML) was stirred at 85° C. under N₂ atmosphere for 12 h. The reaction was quenched with methanol and concentrated to a crude residue which was applied to a column of silica gel eluted with hexane-ethyl acetate (4:1) to give the title compound as a syrup.

¹H NMR (CDCl₃, 200 MHz): δ 7.37-7.22 (m, 10H, ArH), 5.74 (d, 1H, J=4.0Hz, H-1), 4.77 (d, 1H, J_{gem}=12.2Hz, OCH_{A}Ph), 4.62 (dd, 1H, H-2), 4.56 (d, 1H, J_{gem}=12.2Hz, OCH_{B}Ph), 4.52 (d, 1H, J_{gem}=12.2Hz, OCH_{A′}Ph), 4.43 (d, 1H, J_{gem}=12.2Hz, OCH_{B′}Ph), 3.38 (d, 1H, J=10.4Hz, CHOBn), 3.23 (d, 1H, J=10.4Hz, CHO′Bn), 1.63 (s, 3H, CH₃), 1.40 (s, 3H, 4-CH₃), CH₃), 1.34 (s, 3H, CH₃).

¹³C NMR (CDCl₃, 50 MHz): δ 138.42, 138.30, 128.58, 128.01, 127.78, 113.34 (ketal carbon), 104.07 (C-1), 85.70, 79.44, 75.13, 73.64, 72.56, 26.95 (CH₃), 26.37 (CH₃), 19.39 (4-CH₃).

Step J: 1-O-Methyl-3,5-di-O-benzyl-4-C-methylribofuranose

A solution of 1,2-O-isopropylidene-3,5-di-O-benzyl-4-C-methyl-α-D-ribofuranose (1.9 g) in 0.5% HCl-methanol solution was stirred overnight at room temperature. The reaction mixture was quenched with triethylamine and concentrated to a crude residue which was applied to a column of silica gel eluted with hexane-ethyl acetate (4:1) to give the title compound.

¹H NMR (CDCl₃, 200 MHz): δ 7.46-7.39 9 (m, 10H, ArH), 4.88 (s, 1H, H-1), 4.70-4.65 (m, 3H, H-2, OCH₂Ph), 4.15 (s, 2H, OCH₂Ph), 3.49 (s, 2H, H-5a, H-5b), 3.37 (s, 3H, OCH₃), 2.77 (s, 1H, 2-OH), 1.43 (s, 3H, 4-CH₃).

Step K: 1-O-Methyl-2,3,5-tri-O-acetyl-4-C-methylribofuranose

A solution of 1-O-methyl-3,5-di-O-benzyl-4-C-methylribofuranose (9.56 g), 10% Pd—C (9.56 g) in a mixture of dichloromethane (20 mL), methanol (20 mL) and acetic acid (20 mL) was stirred at room temperature under H₂ atmosphere for 12 h. Additional portion of Pd—C (2.1 g) was added and the stirring was continued at the same temperature for total of 24 h. The solids were filtered off and the organic layer was concentrated to a crude residue which was applied to a column of silica gel eluted with dichloromethane-methanol (10:1) to give a compound which was further treated with Ac₂O-pyridine (30 mL, 1:1) in the presence of 4-dimethylaminopyridine (DMAP) (380 mg) at room temperature overnight. The reaction mixture was concentrated to a crude residue, which was passed through a column of silica gel eluted with hexane-ethyl acetate (1:1) to give the title compound as a syrup.

¹H NMR (CDCl₃, 200 MHz, (α-form): δ 5.40 (d, 1H, J=5.3 Hz, H-3), 5.27 (dd, 1.0 Hz, J=1.0 Hz, J=5.3 Hz, H-2) 4.89 (d, 1H, J=0.8 Hz, H-1), 4.21 (d, 1H, J=11.4 Hz, H-5a), 3.98 (d, 1H, J=11.4 Hz, H-5b), 3.36 (s, 3H, OCH₃), 2.11 (s, 3H, Ac), 2.09 (s, 3H, Ac), 2.08 (s, 3H, Ac), 1.29 (s, 3H, 4′-CH₃).

¹³C NMR (CDCl₃, 50 MHz): δ 170.77 (Ac), 169.71 (C═O), 169.66 (C═O), 105.35 (C-1), 83.09, 75.84, 72.66, 69.12, 55.40, 21.03 (Ac), 20.83 (Ac), 20.69 (Ac), 19.94 (4′-CH₃).

Step L: 1,2,3,5-Tetra-O-acetyl-4-C-methylribofuranose

Concentrated sulfuric acid (1.59 mL) in anhydrous acetic anhydride (69 mL) was added dropwise to a cold solution of 1-O-methyl-2,3,5-tri-O-acetyl-4-C-methylribofuranose (6.23 g, 20.5 mmol) in anhydrous acetic anhydride (69 mL) cooled in a ice-bath. The reaction mixture was stirred at the same temperature for 1 h. The reaction was neutralized with a cold saturated aqueous sodium bicarbonate solution (pH=7) and extracted with ethyl acetate, washed saturated aqueous NaHCO₃ solution, saturated aqueous NaCl solution, and dried (MgSO₄). The organic layer was concentrated to a crude residue which was applied to a column of silica gel eluted with hexane-ethyl acetate (4:1) to give the title compound as a syrup.

¹H NMR (CD₂Cl₂, 200 MHz, α-form): δ 5.92 (d, 1H, H-1), 5.46 (d, 1H, H-3), 5.02 (dd, 1H, H-2), 4.30 (d, 1H, J=12.2 Hz, H-5a), 3.84 (d, 1H, J=12.2 Hz, H-5b), 2.12 (s, 3H, Ac), 2.11 (s, 3H, Ac), 2.06 (s, 3H, Ac), 2.02 (s, 3H, AC), 1.61 (s, 3H, 4-CH₃).

Step M: N⁴-Benzoyl-2,3,5-tri-O-acetyl-4′-C-methylcytidine

N,O-bis(trimethylsilyl)acetamide (BSA) (360 μL, 1.46 mmol) was added to a suspension of N⁴-benzoylcytosine (88 mg, 0.411 mmol) in dry acetonitrile (5 mL) and stirred at 85° C. under N₂ atmosphere for 3 h. The mixture was cooled to room temperature, and 1,2,3,5-tetra-O-acetyl-4-C-methylribofuranose (62 mg, 0.187 mmol) in 1,2-dichloroethane (1 mL) and trimethylsilyl trifluoromethanesulfonate (TMSOTf) (33 μL) were added respectively. The reaction mixture was stirred at 65° C. under N₂ atmosphere for 12 h. The mixture was concentrated and the crude residue was applied to a column of silica gel eluted with dichloromethane-methanol (30:1) to give the title compound as an amorphous solid.

¹H NMR (CDCl₃, 200 MHz): δ 9.00 (br, 1H, NHBz), 8.02-7.89 (m, 3H, H-6, ArH), 7.66-7.28 (m, 4H, H-5, ArH), 6.27 (d, 1H, J=4.8 Hz, H′-1), 5.54-5.43 (m, 2H, H′-2, H′-2, H′-3), 4.22 (dd, 2H, J_{gem=}12.2 Hz, H′-5a, H′-5b), 2.18 (s, 3H, Ac), 2.14 (s, 3H, Ac), 2.10 (s, 3H, Ac), 1.39 (s, 3H, 4′-CH₃).

¹³C NMR (CDCl₃, 50 MHz): δ 170.19 (C═O), 169.67 (C═O), 169.58 (C═O), 162.63, 143.95, 133.47, 129.24, 127.81, 97.45 (C-1), 87.68, 84.46, 74.52, 71.03, 67.69, 21.04 (Ac), 20.67 (Ac), 20.62 (Ac), 18.96 (4′-CH₃).

Step N: 4′-C-Methylcytidine

N⁴-Benzoyl-2,3,5-tri-O-acetyl-4-C-methylcytidine was treated with methanolic ammonia at room temperature overnight. The reaction mixture was concentrated to a crude residue which was applied to a column of silica gel eluted with dichloromethane-methanol (4:1) to give the title compound as a white solid.

¹H NMR (CD₃OD, g-DQCOSY, 200 MHz): δ 7.92 (d, 1H, J=7.4 Hz, H-6), 5.83-5.79 (m, 2H, H′-1, H-5), 4.23 (t, 1H, J=5.8 Hz, J=5.4 Hz, H′-2), 4.06 (d, 1H, J=5.6 Hz, H′-3), 3.47 (d, 2H, H′-5a, H′-5b), 1.15 (s, 3H, 4′-CH₃).

¹H NMR (DMSO-d₆, 200 MHz): δ 7.79 (d, 1H, J=7.4 Hz, H-6), 7.12 (br, 2H, NH₂), 5.80 (d, 1H, J=6.4 Hz, H′-1), 5.69 (d, 1H, J=7.4 Hz, H-5), 5.13-5.06 (m, 2H), 4.89 (d, 1H, J=5.2 Hz), 4.15-4.03 (m, 1H), 3.90 (t, 1H, J=5.2 Hz, J=5.6 Hz), 3.41-3.34 (m, 2H), 1.06 (s, 1.06 (s, 3H, 4′-CH₃).

¹³C NMR (CD₃OD, 50 MHz): δ 166.44 (C═O), 157.59, 142.41, 94.94 (C-1), 90.46, 87.22, 75.34, 71.49, 66.87, 17.51 (4′-CH₃).

ESIMS (negative ion mode) (m/z): 256 [M−1]⁻, 292 [M+Cl]⁻

ESIMS (positive ion mode) (m/z): 258 [M+1]⁺, 515 [2M+1]⁺

### EXAMPLE 2

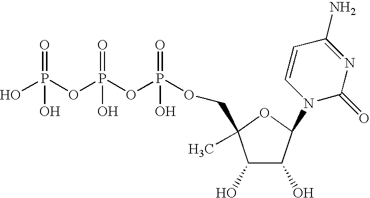

### 4′-C-Methylcytidine-5′-triphosphate

A solution of 4′-C-methylcytidine (15.8 mg) in dry trimethyl phosphate (500 μL) was stirred overnight at room temperature under N₂ atmosphere. The mixture was then cooled with ice-bath (0° C.) and phosphorous oxychloride (8 μL) was added and stirred at the same temperature for 4 h. Tributylamine (73 μL), tributylammonium pyrophosphate (139 mg) and acetonitrile (360 μL) were added and the stirring was continued at 0° C. for 1.5 h. The reaction mixture was then quenched with TEAB solution (0.6 mL) and purified by anion-exchange column.

ESIMS (negative ion mode) (m/z): 496 [M−1]⁻.

### EXAMPLE 3

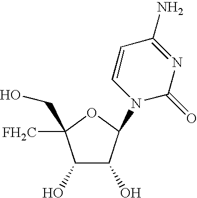

### 4′-C-(Fluoromethyl)cytidine

Step A: 1,2,3,5-Tetra-O-acetyl-4-C-(fluoromethyl)ribofuranose

A solution of 1,2-di-O-acetyl-3,5-di-O-benzyl-4-C-(fluoromethyl)-α-D-ribofuranose [for preparation, see Tetrahedron, 53: 13315 (1997)] (912 mg), 10% Pd—C (912 mg) in a mixture of dichloromethane-methanol (10 mL, 1:1) was stirred under a H₂ atmosphere. After 3 h at room temperature, an additional portion of Pd—C (450 mg) was added, and stirring was continued at room temperature for total of 6 h. The solids were filtered off and the organic layer was concentrated to a crude residue, which was treated overnight with Ac₂O-pyridine (8 mL, 1:1) in the presence of DMAP (4 mg) at room temperature. The mixture was concentrated and the residue applied to a column of silica gel eluted with hexane-ethyl acetate (1:1) to give the title compound as a syrup.

¹H NMR (CD₂Cl₂, 200 MHz): δ 6.15 (s, 1H, H-1), 5.55 (dd, 1H, J=0.8 Hz, J=5.3 Hz, H-2), 5.45 (d, 1H, J=5.2 Hz, H-3), 4.69 (dd, 1H, ), 4.46 (dd, 1H), 4.28 (m, 2H), 2.12)(s, 3H, 2.09 (s, 3H, Ac), 2.07 (s, 3H, Ac), 2.06 (s, 3H, Ac).

¹³C NMR (CD₂Cl₂, 50 MHz): δ 170.17 (C═O), 169.37 (C═O), 169.03 (C═O), 97.13 (C-1), 83.39, 79.92, 74.54, 71.75, 64.29, 21.00 (Ac), 20.67 (Ac), 20.52 (Ac), 20.25 (Ac).

Step B: N⁴-Acetyl-2,3,5-tri-O-acetyl-4′-C-(fluoromethyl)cytidine

N,O-bis(trimethylsilyl)acetamide (490 μL) was added to a suspension of N⁴-acetylcytosine (154 mg, 1.00 mmol) in dry acetonitrile (5 mL) and stirred at 85° C. under N₂ atmosphere for 3 h. The mixture was cooled to room temperature, and 1,2,3,5-tetra-O-acetyl-4-C-(fluoromethyl)ribofuranose (175 mg, 0.5 mmol) in dry 1,2-dichloroethane (1 mL) and TMSOTf (88 μL) were added respectively. The reaction mixture was stirred at 65° C. under N₂ atmosphere for 12 h. An additional portion of TMSOTf (88 uL) was added and the stirring was continued for a total of 24 h. The reaction mixture was concentrated to a crude residue which was applied to a column of silica gel eluted with dichloromethane-methanol (20:1) to give the title compound as an amorphous solid.

¹H NMR (CD₂Cl₂, 200 MHz): δ 10.12 (br, 1H, NHAc), 7.87 (d, 1H, J=7.6 Hz, H-6), 7.46 (d, 1H, J=7.6 Hz, H-5), 6.11 (d, 1H, J=4.8 Hz, H′-1), 5.68 (d, 1H, J=6.4 Hz, H′-3), 5.58(dd, 1H, J=5.0 Hz, J=6.0 Hz, H′-2), 4.76 (dd, 1H, CH_{A}F), 4.53 (dd, 1H, CH_{B}F), 4.45 (dd, 1H, ⁴J_{H,F}=2.0 Hz, J_{gem}=12.0 Hz, H′-5a), 4.32 (dd, 1H, ⁴J_{H,F}=1.8 Hz, J_{gem}=12.0 Hz, H′-5b), 2.24 (s, 3H, NAc), 2.13 (s, 3H, Ac), 2.12 (s, 3H, Ac), 2.06 (s, 3H, Ac).

¹³C NMR (CD₂Cl₂, 50 MHz): δ 171.32 (C═O), 170.12 (C═O), 169.62 (C═O), 169.22 (C═O), 163.72, 154.99, 145.08, 97.41, 90.12, 84.77, 84.42, 83.55, 80.06, 73.86, 70.92, 64.20, 64.08, 24.90 (NAc), 20.79 (Ac), 20.42 (Ac), 20.32 (Ac).

¹⁹F NMR (CD₃OD, 188 MHz): δ −4.54 (t, ²J_{H,F}=49 Hz).

Step C: 4′-C-(Fluoromethyl)cytidine

N⁴-Acetyl-2,3,5-tri-O-acetyl-4′-C-(fluoromethyl)cytidine (65 mg, 0.15 mmol) was treated overnight with methanolic ammonia (6 mL) at room temperature. The reaction mixture was concentrated to a crude residue which was purified on a column of silica gel eluted with dichloromethane-methanol (5:1) to give the title compound as an amorphous solid.

¹H NMR (DMSO-d₆, 200 MHz): δ 7.74 (d, 1H, H-6, J=7.4 Hz, H-6), 7.18 (br, 2H, NH₂), 5.93 (d, 1H, J=7.4 Hz, H′-1), 5.74 (d, 1H, J=7.4 Hz, H-5), 5.27-5.19 (m, 3H), 4.63 (dd, 1H, ²J_{H,H}=10.0 Hz, ²J_{H, F}=21.4 Hz, CH_{A}F), 4.39 (dd, 1H, J_{H,H}=10.0 Hz, ²J_{H,F=}21.4 Hz, CH_{B}F), 4.24-4.05 (m, 3H), 3.52 (dd, 2H, CH₂O).

¹³C NMR (CD₃OD, 200 MHz): δ 166.15, 156.31, 142.42, 95.15, 88.04, 86.28, 85.96, 82.26, 73.63, 71.79, 62.53, 49.28.

¹⁹F NMR (CD₃OD, 188 MHz): δ −5.95 (t, ²J_{H,F}=48.9 Hz).

ESIMS (positive ion mode) (m/z): 276 [M], 282 [M+Na]⁺, 551 [2M]⁺, 573 [2M+Na]⁺

### Biological Assays

The assays employed to measure the inhibition of HCV NS5B polymerase and HCV replication are described below.

The effectiveness of the compounds of the present invention as inhibitors of HCV NS5B RNA-dependent RNA polymerase (RdRp) was measured in the following assay.

A. Assay for Inhibition of HCV NS5B Polymerase:

This assay was used to measure the ability of the nucleoside derivatives of the present invention to inhibit the enzymatic activity of the RNA-dependent RNA polymerase (NS5B) of the hepatitis C virus (HCV) on a heteromeric RNA template.

Procedure:

Assay Buffer Conditions: (50 μL -total/reaction)

20 mM Tris, pH 7.5

50 μM EDTA

5 mM DTT

2 mM MgCl₂

80 mM KCl

0.4 U/μL RNAsin (Promega, stock is 40 units/μL)

0.75 μg t500 (a 500-nt RNA made using T7 runoff transcription with a sequence from the NS2/3 region of the hepatitis C genome)

1.6 μg purified hepatitis C NS5B (form with 21 amino acids C-terminally truncated)

1 μM A,C,U,GTP (Nucleoside triphosphate mix)

[alpha-³²P]-GTP or [alpha-³³P]-GTP

The compounds were tested at various concentrations up to 100 μM final concentration.

An appropriate volume of reaction buffer was made including enzyme and template t500. Nucleoside derivatives of the present invention were pipetted into the wells of a 96-well plate. A mixture of nucleoside triphosphates (NTP's), including the radiolabeled GTP, was made and pipetted into the wells of a 96-well plate. The reaction was initiated by addition of the enzyme-template reaction solution and allowed to proceed at room temperature for 1-2 h.

The reaction was quenched by addition of 20 μL 0.5M EDTA, pH 8.0. Blank reactions in which the quench solution was added to the NTPs prior to the addition of the reaction buffer were included.

50 μL of the quenched reaction were spotted onto DE81 filter disks (Whatman) and allowed to dry for 30 min. The filters were washed with 0.3 M ammonium formate, pH 8 (150 mL/wash until the cpm in 1 mL wash is less than 100, usually 6 washes). The filters were counted in 5-mL scintillation fluid in a scintillation counter.

The percentage of inhibition was calculated according to the following equation:

% Inhibition=[1−(cpm in test reaction−cpm in blank)/(cpm in control reaction−cpm in blank)]×100.

Representative compounds tested in the HCV NS5B polymerase assay exhibited IC₅₀'s less than 100 micromolar.

B. Assay for Inhibition of HCV RNA Replication:

The compounds of the present invention were also evaluated for their ability to affect the replication of Hepatitis C Virus RNA in cultured hepatoma (HuH-7) cells containing a subgenomic HCV Replicon. The details of the assay are described below. This Replicon assay is a modification of that described in V. Lohmann, F. Korner, J-O. Koch, U. Herian, L. Theilmann, and R. Bartenschlager, “Replication of a Sub-genomic Hepatitis C Virus RNAs in a Hepatoma Cell Line,” Science 285:110 (1999).

Protocol:

The assay was an in situ Ribonuclease protection, Scintillation Proximity based-plate assay (SPA). 10,000-40,000 cells were plated in 100-200 μL of media containing 0.8 mg/mL G418 in 96-well cytostar plates (Amersham). Compounds were added to cells at various concentrations up to 100 μM in 1% DMSO at time 0 to 18 h and then cultured for 24-96 h. Cells were fixed (20 min, 10% formalin), permeabilized (20 min, 0.25% Triton X-100/PBS) and hybridized (overnight, 50° C.) with a single-stranded ³³P RNA probe complementary to the (+) strand NS5B (or other genes) contained in the RNA viral genome. Cells were washed, treated with RNAse, washed, heated to 65° C. and counted in a Top-Count. Inhibition of replication was read as a decrease in counts per minute (cpm).

Human HuH-7 hepatoma cells, which were selected to contain a subgenomic replicon, carry a cytoplasmic RNA consisting of an HCV 5′ non-translated region (NTR), a neomycin selectable marker, an EMCV IRES (internal ribosome entry site), and HCV non-structural proteins NS3 through NS5B, followed by the 3′ NTR.

Representative compounds tested in the replication assay exhibited EC₅₀'s less than 100 micromolar.

The nucleoside derivatives of the present invention were also evaluated for cellular toxicity and anti-viral specificity in the counterscreens described below.

C. Counterscreens:

The ability of the nucleoside derivatives of the present invention to inhibit human DNA polymerases was measured in the following assays.

a. Inhibition of Human DNA Polymerases Alpha and Beta:

Reaction Conditions:

50 μL reaction volume

Reaction Buffer Components:

20 mM Tris-HCl, pH 7.5

200 μg/mL bovine serum albumin

100 mM KCl

2 mM β-mercaptoethanol

10 mM MgCl₂

1.6 μM dA, dG, dC, dTTP

α-³³P-dATP

Enzyme and template:

0.05 mg/mL gapped fish sperm DNA template

0.01 U/μL DNA polymerase α or β

Preparation of Gapped Fish Sperm DNA Template:

Add 5 μL 1M MgCl₂ to 500 μL activated fish sperm DNA (USB 70076);

Warm to 37° C. and add 30 μAL of 65 U/μL of exonuclease III (GibcoBRL 18013-011);

Incubate 5 min at 37° C.;

Terminate reaction by heating to 65° C. for 10 min;

Load 50-100 μL aliquots onto Bio-spin 6 chromatography columns (Bio-Rad 732-6002) equilibrated with 20 mM Tris-HCl, pH 7.5;

Elute by centrifugation at 1,000×g for 4 min;

Pool eluate and measure absorbance at 260 nm to determine concentration.

The DNA template was diluted into an appropriate volume of 20 mM Tris-HCl, pH 7.5 and the enzyme was diluted into an appropriate volume of 20 mM Tris-HCl, containing 2 mM β-mercaptoethanol, and 100 mM KCl. Template and enzyme were pipetted into microcentrifuge tubes or a 96 well plate. Blank reactions excluding enzyme and control reactions excluding test compound were also prepared using enzyme dilution buffer and test compound solvent, respectively. The reaction was initiated with reaction buffer with components as listed above. The reaction was incubated for 1 hour at 37° C. The reaction was quenched by the addition of 20 μL 0.5M EDTA. 50 μL of the quenched reaction was spotted onto Whatman DE81 filter disks and air dried. The filter disks were repeatedly washed with 150 mL 0.3M ammonium formate, pH 8 until 1 mL of wash is <100 cpm. The disks were washed twice with 150 mL absolute ethanol and once with 150 mL anhydrous ether, dried and counted in 5 mL scintillation fluid.

The percentage of inhibition was calculated according to the following equation:

% inhibition=[1−(cpm in test reaction−cpm in blank)/(cpm in control reaction−cpm in blank)]×100.

b. Inhibition of Human DNA Polymerase Gamma:

The potential for inhibition of human DNA polymerase gamma was measured in reactions that included 0.5 ng/μL enzyme; 10 μM dATP, dGTP, dCTP, and TTP; 2 μCi/reaction [α-³³P]-dATP, and 0.4 μg/μL activated fish sperm DNA (purchased from US Biochemical) in a buffer containing 20 mM Tris pH8, 2 mM β-mercaptoethanol, 50 mM KCl, 10 mM MgCl₂, and 0.1 μg/μL BSA. Reactions were allowed to proceed for 1 h at 37° C. and were quenched by addition of 0.5 M EDTA to a final concentration of 142 mM. Product formation was quantified by anion exchange filter binding and scintillation counting. Compounds were tested at up to 50 μM.

The percentage of inhibition was calculated according to the following equation:

% inhibition=[1−(cpm in test reaction−cpm in blank)/(cpm in control reaction−cpm in blank)]×100.

The ability of the nucleoside derivatives of the present invention to inhibit HIV infectivity and HIV spread was measured in the following assays.

c. HIV Infectivity Assay

Assays were performed with a variant of HeLa Magi cells expressing both CXCR4 and CCR5 selected for low background β-galactosidase (β-gal) expression. Cells were infected for 48 h, and β-gal production from the integrated HIV-1 LTR promoter was quantified with a chemiluminescent substrate (Galactolight Plus, Tropix, Bedford, Mass.). Inhibitors were titrated (in duplicate) in twofold serial dilutions starting at 100 μM; percent inhibition at each concentration was calculated in relation to the control infection.

d. Inhibition of HIV Spread

The ability of the compounds of the present invention to inhibit the spread of the human immunedeficiency virus (HIV) was measured by the method described in U.S. Pat. No. 5,413,999 (May 9, 1995), and J. P. Vacca, et al., Proc. Natl. Acad. Sci., 91: 4096-4100 (1994), which are incorporated by reference herein in their entirety.

The nucleoside derivatives of the present invention were also screened for cytotoxicity against cultured hepatoma (HuH-7) cells containing a subgenomic HCV Replicon in an MTS cell-based assay as described in the assay below. The HuH-7 cell line is described in H. Nakabayashi, et al., Cancer Res., 42: 3858 (1982).

e. Cytotoxicity Assay:

Cell cultures were prepared in appropriate media at concentrations of approximately 1.5×10⁵ cells/mL for suspension cultures in 3 day incubations and 5.0×10⁴ cells/mL for adherent cultures in 3 day incubations. 99 μL of cell culture was transferred to wells of a 96-well tissue culture treated plate, and 1 μL of 100-times final concentration of the test compound in DMSO was added. The plates were incubated at 37° C. and 5% CO₂ for a specified period of time. After the incubation period, 20 μL of CellTiter 96 Aqueous One Solution Cell Proliferation Assay reagent (MTS) (Promega) was added to each well and the plates were incubated at 37° C. and 5% CO₂ for an additional period of time up to 3 h. The plates were agitated to mix well and absorbance at 490 nm was read using a plate reader. A standard curve of suspension culture cells was prepared with known cell numbers just prior to the addition of MTS reagent. Metabolically active cells reduce MTS to formazan. Formazan absorbs at 490 nm. The absorbance at 490 nm in the presence of compound was compared to absorbance in cells without any compound added.

Reference: Cory, A. H. et al., “Use of an aqueous soluble tetrazolium/formazan assay for cell growth assays in culture,” Cancer Commun. 3: 207 (1991).

The following assays were employed to measure the activity of the compounds of the present invention against other RNA-dependent RNA viruses:

a. Determination of In Vitro Antiviral Activity of Compounds Against Rhinovirus (Cytopathic Effect Inhibition Assay):

Assay conditions are described in the article by Sidwell and Huffman, “Use of disposable microtissue culture plates for antiviral and interferon induction studies,” Appl. Microbiol. 22: 797-801 (1971).

Viruses:

Rhinovirus type 2 (RV-2), strain HGP, was used with KB cells and media (0.1% NaHCO₃, no antibiotics) as stated in the Sidwell and Huffman reference. The virus, obtained from the ATCC, was from a throat swab of an adult male with a mild acute febrile upper respiratory illness. Rhinovirus type 9 (RV-9), strain 211, and rhinovirus type 14 (RV-14), strain Tow, were also obtained from the American Type Culture Collection (ATCC) in Rockville, Md. RV-9 was from human throat washings and RV-14 was from a throat swab of a young adult with upper respiratory illness. Both of these viruses were used with HeLa Ohio-1 cells (Dr. Fred Hayden, Univ. of VA) which were human cervical epitheloid carcinoma cells. MEM (Eagle's minimum essential medium) with 5% Fetal Bovine serum (FBS) and 0.1% NaHCO₃ was used as the growth medium.

Antiviral test medium for all three virus types was MEM with 5% FBS, 0.1% NaHCO₃, 50 μg gentamicin/mL, and 10 mM MgCl₂.

2000 μg/mL was the highest concentration used to assay the compounds of the present invention. Virus was added to the assay plate approximately 5 min after the test compound. Proper controls were also run. Assay plates were incubated with humidified air and 5% CO₂ at 37° C. Cytotoxicity was monitored in the control cells microscopically for morphologic changes. Regression analysis of the virus CPE data and the toxicity control data gave the ED50 (50% effective dose) and CC₅₀ (50% cytotoxic concentration). The selectivity index (SI) was calculated by the formula: SI=CC50÷ED50.

b. Determination of In Vitro Antiviral Activity of Compounds Against Dengue, Banzi, and Yellow Fever (CPE Inhibition Assay)

Assay details are provided in the Sidwell and Huffman reference above.

Viruses:

Dengue virus type 2, New Guinea strain, was obtained from the Center for Disease Control. Two lines of African green monkey kidney cells were used to culture the virus (Vero) and to perform antiviral testing (MA-104). Both Yellow fever virus, 17D strain, prepared from infected mouse brain, and Banzi virus, H 336 strain, isolated from the serum of a febrile boy in South Africa, were obtained from ATCC. Vero cells were used with both of these viruses and for assay.

Cells and Media:

MA-104 cells (BioWhittaker, Inc., Walkersville, Md.) and Vero cells (ATCC) were used in Medium 199 with 5% FBS and 0.1% NaHCO₃ and without antibiotics.

Assay medium for dengue, yellow fever, and Banzi viruses was MEM, 2% FBS, 0.18% NaHCO₃ and 50 μg gentamicin/mL.

Antiviral testing of the compounds of the present invention was performed according to the Sidwell and Huffman reference and similar to the above rhinovirus antiviral testing. Adequate cytopathic effect (CPE) readings were achieved after 5-6 days for each of these viruses.

c. Determination of In Vitro Antiviral Activity of Compounds Against West Nile Virus (CPE Inhibition Assay)

Assay details are provided in the Sidwell and Huffman reference cited above. West Nile virus, New York isolate derived from crow brain, was obtained from the Center for Disease Control. Vero cells were grown and used as described above. Test medium was MEM, 1% FBS, 0.1% NaHCO₃ and 50 μg gentamicin/mL.

Antiviral testing of the compounds of the present invention was performed following the methods of Sidwell and Huffman which are similar to-those used to assay for rhinovirus activity. Adequate cytopathic effect (CPE) readings were achieved after 5-6 days.

d. Determination of In Vitro Antiviral Activity of Compounds Against Rhino, Yellow Fever, Dengue, Banzi, and West Nile Viruses (Neutral Red Uptake Assay)

After performing the CPE inhibition assays above, an additional cytopathic detection method was used which is described in “Microtiter Assay for Interferon: Microspectrophotometric Quantitation of Cytopathic Effect,” Appl. Environ. Microbiol. 31: 35-38 (1976). A Model EL309 microplate reader (Bio-Tek Instruments Inc.) was used to read the assay plate. ED50's and CD50's were calculated as above.

### Example of A Pharmaceutal Formulation

As a specific embodiment of an oral composition of a compound of the present invention, 50 mg of the compound of Example 1 or Example 3 is formulated with sufficient finely divided lactose to provide a total amount of 580 to 590 mg to fill a size O hard gelatin capsule.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for severity of the HCV infection. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

What is claimed is:

1. A compound of the structural formula I 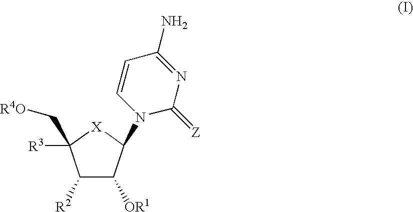
or a pharmaceutically acceptable salt thereof; wherein
X is O or S;
Z is O or S;
R¹ is hydrogen, methyl, C₁₋₁₆ alkylcarbonyl, C₂₋₁₈ alkenylcarbonyl, C₁₋₁₀ alkyloxycarbonyl, C₃₋₆ cycloalkylcarbonyl, C₃₋₆ cycloalkyloxycarbonyl, CH₂O(C═O)C₁₋₄ alkyl, CH(C₁₋₄ alkyl)O(C═O)C₁₋₄ alkyl, or an amino acyl residue of structural formula 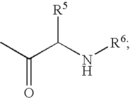
R² is hydrogen, hydroxy, fluoro, amino, methyl, methoxy, C₁₋₁₆ alkylcarbonyloxy, C₂₋₁₈ alkenylcarbonyloxy, C₁₋₁₀ alkyloxycarbonyloxy, C₃₋₆ cycloalkylcarbonyloxy, C₃₋₆ cycloalkyloxycarbonyloxy, —OCH₂O(C═O)C₁₋₄ alkyl, —OCH(C₁₋₄ alkyl)O(C═O)C₁₋₄ alkyl, or an amino acyloxy residue of structural formula 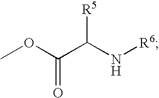
R³ is selected from the group consisting of methyl, ethynyl, cyano, aminocarbonyl, fluoromethyl, bromomethyl, trifluoromethyl, aminomethyl, fluoro, chloro, and bromo;
R⁴ is hydrogen, C₁₋₁₀ alkylcarbonyl, C₂₋₁₈ alkenylcarbonyl, C₁₋₁₀ alkyloxycarbonyl, C₃₋₆ cycloalkylcarbonyl, C₃₋₆ cycloalkyloxycarbonyl, CH₂O(C═O)C₁₋₄ alkyl, CH(C₁₋₄ alkyl)O(C═O)C₁₋₄ alkyl, P₃O₉H₄, P₂O₆H₃, P(O)R⁷R⁸, or an amino acyl residue of structural formula 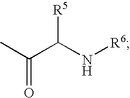
R⁵ is hydrogen, C₁₋₄ alkyl, or phenyl C₀₋₂ alkyl;
R⁶ is hydrogen, C₁₋₄ alkyl, C₁₋₄ acyl, benzoyl, C₁₋₄ alkyloxycarbonyl, phenyl C₀₋₂ alkyloxycarbonyl, C₁₋₄ alkylaminocarbonyl, phenyl C₀₋₂ alkylaminocarbonyl, C₁₋₄ alkylsulfonyl, or phenyl C₀₋₂ alkylsulfonyl; and
R⁷ and R⁸ are each independently hydroxy, OCH₂CH₂SC(═O)C₁₋₄ alkyl, OCH₂O(C═O)OC₁₋₄ alkyl, NHCHMeCO₂Me, OCH(C₁₋₄ alkyl)O(C═O)C₁₋₄ alkyl, 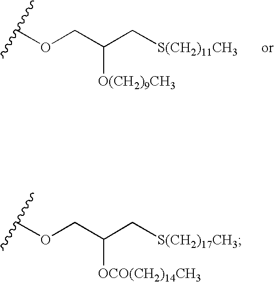
with the proviso that when X and Z are O, R¹ and R⁴ are hydrogen, and R² is hydroxy, then R³ is not methyl, fluoromethyl, ethynyl, or cyano.

2. The compound of claim 1 wherein X and Z are O.

3. The compound of claim 1 wherein
R¹ is hydrogen, C₁₋₁₆ alkylcarbonyl, or an aminoacyl residue of structural formula 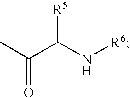
R² is hydroxy, C₁₋₁₆ alkylcarbonyloxy, or an amino acyloxy residue of structural formula 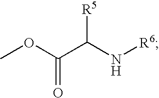
R³ is methyl or fluoromethyl; and
R⁴ is hydrogen, C₁₋₁₀ alkylcarbonyl, P₃O₉H₄, or an amino acyl residue of structural formula 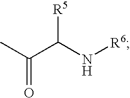
with the proviso that when X and Z are O, R¹ and R⁴ are hydrogen, and R² is hydroxy, then R³ is not methyl, fluoromethyl, ethynyl, or cyano.

4. A pharmaceutical composition comprising a compound of claim 1 and a pharmaceutically acceptable carrier.

5. The pharmaceutical composition of claim 4 useful for inhibiting RNA-dependent RNA viral polymerase, inhibiting RNA-dependent RNA replication, and/or treating RNA-dependent RNA viral infection.

6. The pharmaceutical composition of claim 5 wherein said RNA-dependent RNA viral polymerase is HCV NS5B polymerase, said RNA-dependent RNA viral replication is HCV replication, and said RNA-dependent RNA viral infection is HCV infection.

7. A method of treating RNA-dependent RNA viral infection comprising administering to a mammal in need of such treatment a therapeutically effective amount of a compound of structural formula I: 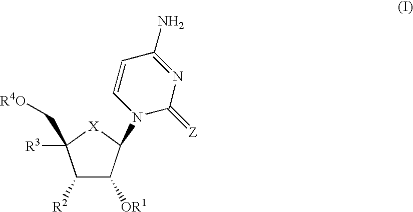
or a pharmaceutically acceptable salt thereof; wherein
X is O or S;
Z is O or S;
R¹ is hydrogen, methyl, C₁₋₁₆ alkylcarbonyl, C₂₋₁₈ alkenylcarbonyl, C₁₋₁₀ alkyloxycarbonyl, C₃₋₆ cycloalkylcarbonyl, C₃₋₆ cycloalkyloxycarbonyl, CH₂O(C═O)C₁₋₄ alkyl, CH(C₁₋₄ alkyl)O(C═O)C₁₋₄ alkyl, or an amino acyl residue of structural formula 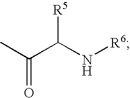
R² is hydrogen, hydroxy, fluoro, amino, methyl, methoxy, C₂₋₁₈ alkenylcarbonyloxy, C₂₋₁₈ alkenylcarbonyloxy, C₁₋₁₀ alkyloxycarbonyloxy, C₃₋₆ cycloalkylcarbonyloxy, C₃₋₆ cycloalkyloxycarbonyloxy, —OCH₂O(C═O)C₁₋₄ alkyl, —OCH(C₁₋₄ alkyl)O(C═O)C₁₋₄ alkyl, or an amino acyloxy residue of structural formula 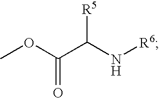
R³ is selected from the group consisting of methyl, ethynyl, cyano, aminocarbonyl, fluoromethyl, bromomethyl, trifluoromethyl, aminomethyl, fluoro, chloro, and bromo;
R⁴ is hydrogen, C₁₋₁₀ alkylcarbonyl, C₂₋₁₈ alkenylcarbonyl, C₁₋₁₀ alkyloxycarbonyl, C₃₋₆ cycloalkylcarbonyl, C₃₋₆ cycloalkyloxycarbonyl, CH₂O(C═O)C₁₋₄ alkyl, CH(C₁₋₄ alkyl)O(C═O)C1-4 alkyl, P₃O₉H₄, P₂O₆H₃, P(O)R⁷R⁸, or an amino acyl residue of structural formula 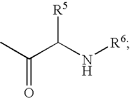
R⁵ is hydrogen, C₁₋₄ alkyl, or phenyl C₀₋₂ alkyl;
R⁶ is hydrogen, C₁₋₄ alkyl, C₁₋₄ acyl, benzoyl, C₁₋₄ alkyloxycarbonyl, phenyl C₀₋₂ alkyloxycarbonyl, C₁₋₄ alkylaminocarbonyl, phenyl C₀₋₂ alkylaminocarbonyl, C₁₋₄ alkylsulfonyl, or phenyl C₀₋₂ alkylsulfonyl; and
R⁷ and R⁸ are each independently hydroxy, OCH₂CH₂SC(═O)C₁₋₄ alkyl, OCH₂O(C═O)OC₁₋₄ alkyl, NHCHMeCO₂Me, OCH(C₁₋₄ alkyl)O(C═O)C₁₋₄ alkyl,

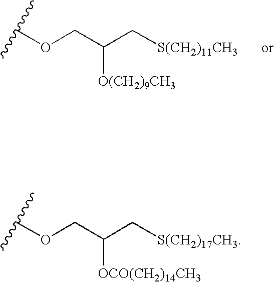

8. The method of claim 7 wherein X and Z are O.

9. The method of claim 8 wherein
R¹ is hydrogen, C₁₋₁₆ alkylcarbonyl, or an aminoacyl residue of structural formula 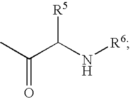
R² is hydroxy, C₁₋₁₆ alkylcarbonyloxy, or an amino acyloxy residue of structural formula 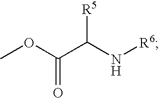
R³ is methyl or fluoromethyl; and
R⁴ is hydrogen, C₁₋₁₀ alkylcarbonyl, P₃O₉H₄, or an amino acyl residue of structural formula

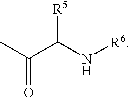

10. The method of claim 9 wherein the compound of formula I is 4′-C-methylcytidine or 4′-C-(fluoromethyl)cytidine; or a pharmaceutically acceptable salt thereof.

11. The method of claim 7 wherein said RNA-dependent RNA viral infection is HCV infection.

12. The method of claim 11 in combination with a therapeutically effective amount of another agent active against HCV.

13. The method of claim 12 wherein said agent active against HCV is ribavirin; levovirin; thymosin alpha-1; interferon-β; an inhibitor of NS3 serine protease; an inhibitor of inosine monophosphate dehydrogenase; intererfon-α or pegylated intererfon-α, alone or in combination with ribavirin or levovirin.

14. The method of claim 13 wherein said agent active against HCV is intererfon-α or pegylated intererfon-α, alone or in combination with ribavirin.

15. A method of treating HCV infection in a mammal in need thereof comprising administering to such mammal a therapeutically effective amount of 4′-C-methylcytidine or a pharmaceutically acceptable salt thereof.
